(19) United States
(12) Patent Application Publication
(10) Pub. No.: US 2010/0104596 A1
(43) Pub. Date: Apr. 29, 2010

(54) ACUTTE TRANSMITTED HIV ENVELOPE SIGNATURES

(75) Inventors: Barton F. Haynes, Durham, NC (US); Betle T. Korber, Oakland, CA (US); Beatrice H. Hahn, Birmingham, AL (US); Tanmoy Bhattacharya, Oakland, CA (US); Gnana Ganakaram, Oakland, CA (US); Feng Gao, Durham, NC (US); Ron Swanstrom, Chapel Hill, NC (US); George Shaw, Birmingham, AL (US); Barton F. Uaynes, Durham, NC (US); Betle T Korber, Oakland, CA (US)

Correspondence Address: NIXON & VANDERHYE, PC, 901 NORTH GLEBE ROAD, 11TH FLOOR, ARLINGTON, VA, 22203, US

(73) Assignees: THE REGENTS OF THE UNIVERSITY OF CALIFORNIA, Oakland, CA (US); DUKE UNIVERSITY, Durham, NC (US); THE UNIVERSITY OF ALABAMA AT BIRMINGHAM, Birmingham, AL (US); UNIVERSITY OF NORTH CAROLINA AT CHAPEL HILL, Chapel Hill, NC (US)

(21) Appl. No.: 12/450,395

(22) PCT Filed: Mar. 27, 2008
(86) PCT No.: PCT/US2008/003965

§ 371 Date: Sep. 24, 2009

Related U.S. Application Data
(60) Provisional application No. 60/907,259, filed on Mar. 27, 2007.

(51) Int. Cl. A61K 39/21; A61P 37/04

(52) U.S. Cl. 424/208.1

(57) ABSTRACT

The present invention relates, in general, to human immunodeficiency virus (HIV) and, in particular, to a method of inducing an immune response to HIV in a patient and to immunogens suitable for use in such a method. The invention also relates to diagnostic test kits and methods of using same.

This application claims priority from U.S. Provisional Application No. 60/907,259, filed Mar. 27, 2007, the entire content of which is incorporated herein by reference.

This invention was made with government support under Grant No. A10678501 awarded by the National Institutes of Health. The government has certain rights in the invention.

### TECHNICAL FIELD

The present invention relates, in general, to human immunodeficiency virus (HIV) and, in particular, to a method of inducing an immune response to HIV in a patient and to immunogens suitable for use in such a method. The invention also relates to diagnostic test kits and methods of using same.

### BACKGROUND

For development of an HIV vaccine, viral diversity remains one of the most difficult problems (Gaschen et al, Science 296:2354 (2002)). Antibodies against the HIV-1 envelope have been shown to be protective when present in high levels early on before infection, and when the antibodies have specificity for the challenge immunodeficiency virus strain (Mascola et al, Nat. Med. 6:207-210 (2000); Mascola et al, J. Virology 73:4009-4018 (1999)). While viral diversity in chronic HIV infection subjects is extraordinarily diverse, viral diversity after HIV-1 transmission is reduced (Zhang et al, J. Virol. 67:33456-3356 (1993); Zhu et al, Science 261:1179-1181 (1993); Ritola et al, J. Virol. 78:11208-11218 (2004)). Rare variants in the donor may be selectively passed to the recipient (Wolinsky et al, Science 255:1134-1137 (2000)).

In acute HIV infection, there is disproportionately greater loss of diversity in HIV-1 envelope compared to gag, suggesting env-mediated viral selection during the transmission event (Zhang et al, J. Virol. 67:33456-3356 (1993); Zhu et al, Science 261:1179-1181 (1993)). Recent data have shown that neutralization sensitive env with shortened variable loops are selectively transmitted during acute HIV infection (Derdeyn et al, Science 303:2019-2022 (2004)). It has also been shown that depletion of B cells during SIV acute infection prevents control of SIV infection (Miller et al, J. Virology e pub Feb. 28, 2007).

The present invention results, at least in part, from the identification of vaccine design criteria which, if fulfilled, can result in an effective vaccine against HIV.

### SUMMARY OF THE INVENTION

The present invention relates generally to HIV. A specific aspect of the invention relates to a method of inducing an immune response to HIV in a patient and to immunogens suitable for use in such a method. A further specific aspect of the invention relates to diagnostic test kits and to methods of using same.

Objects and advantages of the present invention will be clear from the description that follows.

### BRIEF DESCRIPTION OF THE DRAWINGS

FIG. 1. ML tree of Patient consensus 100 bootstraps.

FIG. 2. SGA-derived envelope clones.

FIG. 3. Z20 histogram of hamming distance frequencies.

FIG. 4. Homogeneous Patient 1012.

FIG. 5. Homogeneous Patient 700010058.

FIG. 6. Heterogeneous Patient Z18.

FIG. 7. Heterogeneous Patient SC33.

FIG. 8. Heterogeneous Patients.

FIG. 9. 73 Heterogeneous Patients.

FIG. 10. 27 Patients have complex, multi-peaked distributions ˜15% have Hamming distances suggesting heterogeneous infections.

FIG. 11. SGA derived functional Envelope clones.

FIG. 12. Mutual information signature: each vertical line represents one person, with the number of sequences obtained indicated by the height. The breakdown of amino acids in each position is indicated by the color. Position 11 is more variable in chronics, and tolerates P and N.

FIG. 13. Position 11 in signal peptide.

FIG. 14. NNSSG_E_KMEKG.

FIGS. 15A-15Z. Acute transmission signatures.

### DETAILED DESCRIPTION OF THE INVENTION

The present invention relates to HIV Envs from transmitted viruses that contain the transmission signatures described herein (note particularly the Example that follows) and methods of using same as vaccine immunogens. The invention further relates to HIV Envs from transmitted viruses that contain the indicated transmission signatures for use as diagnostic targets in diagnostic tests. In addition, the invention relates to the HIV Env transmitted signatures incorporated into consensus Envs (that is, the amino acids of a transmitted virus sequence signature can be incorporated into the sequence of an otherwise group M consensus or subtype consensus Env). Further, the invention relates to HIV transmitted virus consensus Envs (with the transmitted virus signatures) and to methods of using same as immunogens. Additionally, the invention relates to the HIV transmitted virus consensus Envs (with the transmitted virus signatures) and to methods of using same as diagnostic targets for tests.

The present invention results, at least in part, from a study made of a series of HIV-1 acute and early transmission patients. Envelope sequences from these patients were compared with control groups of chronically infected patients. A transmission bottle neck has been found in the transmission virus with, in 75% of patients, evidence for one virus species transmitted, and, in about 15% of patients, evidence for multiple strains transmitted (it is believed that the transmitted signature in the Env are involved with which viruses are transmitted). Identification of transmission strain envelope signatures that are characteristic of the transmitted virus but not chronic HIV strains has begun. Described herein are two initial transmitted Env signatures and methods of using these signatures and the transmitted HIV-1 strain database to design effective HIV-1 envelope immunogens for HIV-1 vaccine development.

A vaccine that fulfills the following criteria can be expected to inhibit transmission of HIV efficiently:
1. induces the production of antibodies that bind conserved functional transmitted envelope trimer epitopes;
2. induces antibody production by a B cell population that can respond to infection within hours to days;
3. induces the production of antibodies at mucosal surfaces;
4. induces high titers of antibodies locally at the site of transmission; and
5. prevents or limits massive apoptosis or apoptosis-mediated immune suppression.

The immunogens of the invention can be chemically synthesized and purified using methods which are well known to the ordinarily skilled artisan. The immunogens can also be synthesized by well-known recombinant DNA techniques. Nucleic acids encoding the immunogens of the invention can be used as components of, for example, a DNA vaccine wherein the encoding sequence is administered as naked DNA or, for example, a minigene encoding the immunogen can be present in a viral vector. The encoding sequence can be present, for example, in a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, another pox virus vector, recombinant polio and other enteric virus vector, Salmonella species bacterial vector, Shigella species bacterial vector, Venezuelan Equine Encephalitis Virus (VEE) vector, a Semliki Forest Virus vector, or a Tobacco Mosaic Virus vector. The encoding sequence, can also be expressed as a DNA plasmid with, for example, an active promoter such as a CMV promoter. Other live vectors can also be used to express the sequences of the invention. Expression of the immunogen of the invention can be induced in a patient's own cells, by introduction into those cells of nucleic acids that encode the immunogen, preferably using codons and promoters that optimize expression in human cells. Examples of methods of making and using DNA vaccines are disclosed in, for example, U.S. Pat. Nos. 5,580,859, 5,589,466, and 5,703,055.

The invention includes compositions comprising an immunologically effective amount of the immunogen of the invention, or nucleic acid sequence encoding same, in a pharmaceutically acceptable delivery system. The compositions can be used for prevention and/or treatment of immunodeficiency virus infection. The compositions of the invention can be formulated using adjuvants (e.g., alum, AS021 (from GSK) oligo CpGs, MF59 or Emulsigen), emulsifiers, pharmaceutically-acceptable carriers or other ingredients routinely provided in vaccine compositions. Optimum formulations can be readily designed by one of ordinary skill in the art and can include formulations for immediate release and/or for sustained release, and for induction of systemic immunity and/or induction of localized mucosal immunity (e.g, the formulation can be designed for intranasal administration). The present compositions can be administered by any convenient route including subcutaneous, intranasal, intrarectal, intravaginal, oral, intramuscular, or other parenteral or enteral route, or combinations thereof. The immunogens can be administered in an amount sufficient to induce an immune response, e.g., as a single dose or multiple doses. Optimum immunization schedules can be readily determined by the ordinarily skilled artisan and can vary with the patient, the composition and the effect sought.

Examples of compositions and administration regimens of the invention include consensus or mosaic gag genes and consensus or mosaic nef genes and consensus or mosaic pol genes and consensus Env with transmitted signatures or mosaic Env with transmitted signatures or wild-type transmitted virus Env with transmitted signatures, expressed as, for example, a DNA prime recombinant Vesicular stomatitis virus boost and a recombinant Envelope protein boost for antibody, or DNA prime recombinant adenovirus boost and Envelope protein boost, or, for just antibody induction, only the recombinant envelope as a protein in an adjuvant. (See U.S. application Ser. No. 10/572,638 and PCT/US2006/032907.)

The invention contemplates the direct use of both the immunogen of the invention and/or nucleic acids encoding same and/or the immunogen expressed as minigenes in the vectors indicated above. For example, a minigene encoding the immunogen can be used as a prime and/or boost.

It will be appreciated from a reading of this disclosure that the whole Envelope gene can be used or portions thereof (i.e., as minigenes). In the case of expressed proteins, protein subunits can be used.

In accordance with the invention, the following can be used in HIV vaccine design to achieve the induction of protective antibodies to HIV-1:
1. Immunization with HIV env constructs derived from wild-type transmitted HIV-1 strains containing the transmission signatures set forth in the Example below.
2. Incorporation of these transmitted signatures into consensus HIV-1 Envs that have been developed from chronic HIV-1 sequences, such as CONS (Liao et al, Virology 353:268-82 (2006)), or a newer group m consensus, year 2003 CONT or subtype consensus Envs such as CONA 2003, CONB 2003, or CONC 2003. Later versions of these consensus sequences can be used derived from sequences later than 2003 from the Los Alamos HIV Sequence Database. Other subtype consensus genes can use used as well, such as derived from clades AE_{—}01, AG recombinants, G, F etc.
3. Development of a transmitted isolate env consensus solely based on consensus sequences from individual patients. This requires adding non-B sequences to the transmitted HIV database—these sequences are being generated by the Center for HIV AIDS Vaccine Immunology.
4. Expression of any of the Envs described in the Example may require them to be in the most native conformation. Thus, Envs can be expressed as gp140 C (cleavage mutant) F (fusion domain deleted) forms, as gp140 C forms, as gp160 forms in virus like particles (Sailaja et al, Virology Feb. 2, 2007 e pub.), or as stabilized trimers using GCN4 trimerization motifs at the C termini of the gp140s (Pancera, J. Virol. 79:9954-9969 (2005)).
5. Alternatively, if the transmission signatures confer on the Env stabilized neutralization epitopes, portions of Env containing the stabilized epitopes can be expressed as a subunit and used for immunization.
6. Env recognition by the T cell arm of the immune system is important for HIV vaccine design (Weaver et al, J. Virol. 80:6745-56 (2006)). Thus, wild-type transmitted Envs with these signatures or consensus Envs containing these signatures can stabilize T cell recognition of certain T cell epitopes and be advantageous for T cell vaccine design.
7. T cells recognize immunogenic epitopes throughout the HIV genome (Letvin et al, Nat. Med. 9:861-866 (2003)) and thus inclusion into the transmitted HIV database full genome sequences of transmitted viruses can expedite and make possible the design of full HIV vaccines with T cell epitopes from throughout the HIV genome.

As pointed out above, the invention also relates to diagnostic targets and diagnostic tests. For example, Envelope containing the transmission virus signature can be expressed by transient or stable transfection of mammalian cells (or they can be expressed, for example; as recombinant Vaccinia virus proteins). The protein can be used in ELISA, Luminex bead test, or other diagnostic tests to detect antibodies to the transmitted virus in a biological sample from a patient at the earliest stage of HIV infection.

Certain aspects of the invention can be described in greater detail in the non-limiting Example that follows. (See also U.S. application Ser. No. 10/572,638, filed Dec. 22, 2006 and International Patent Application No. PCT/US2006/032907 filed Aug. 23, 2006.)

### Example

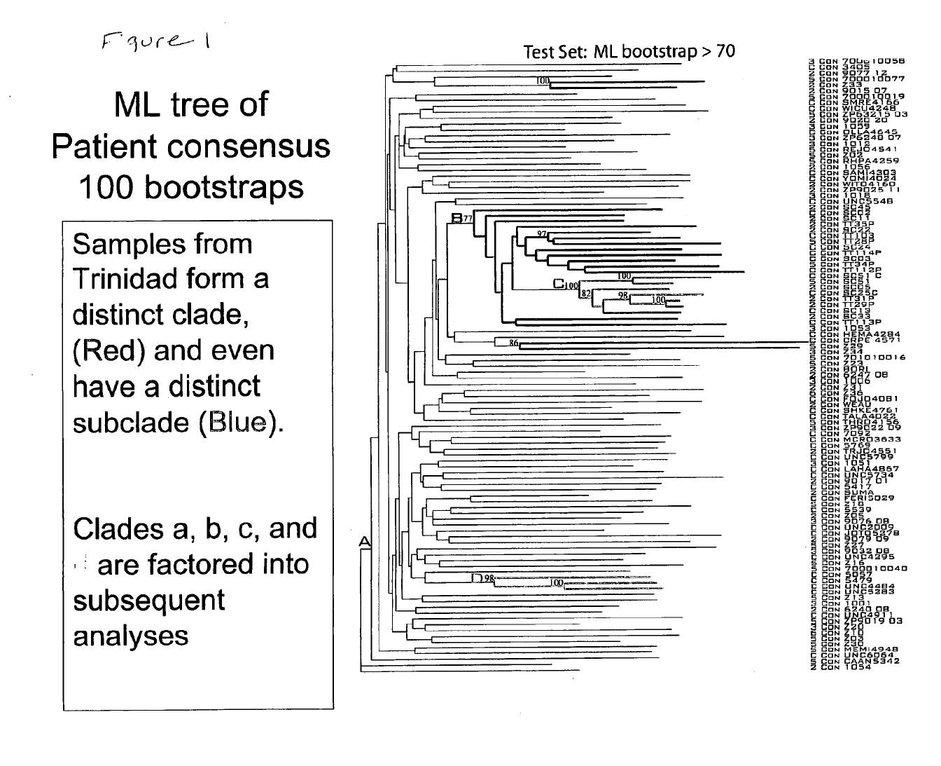

Characterization of the envelope of the HIV-1 transmitted virus is critical to design of an effective envelope based vaccine. 4260 B Glade env sequences from 192 individuals have been codon-aligned, hypermutated sequences or sequences with gaps of greater than 100 bases have been deleted. These sequences have been split into test, validation and early sets. Likelihood trees have been created based on the patient consensus sequences of the sets to look for robust within-subtype B clades: certain samples, in particular, the CHAVI samples from the USA and Trinidad, had distinct geographic lineages evident in the tree (FIG. 1).

The test set consists of 26 Feibig II, acute samples with no detectable HIV specific immunity (Feibig et al, AIDS 17:1871-1875 (2003)), 14 Feibig III, acute HIV infection (AHI) samples that were antibody+, and 40 matched chronic patients. A second set of samples was used for a validation set: again, with 26 Fiebig I-II AHI samples before HIV specific immunity, 14 Feibig III-IV AHI that were antibody positive, and 38 B Glade chronic patients from the Los Alamos Database (Bailey et al, J. Virol. 80:4758-62 (2006))

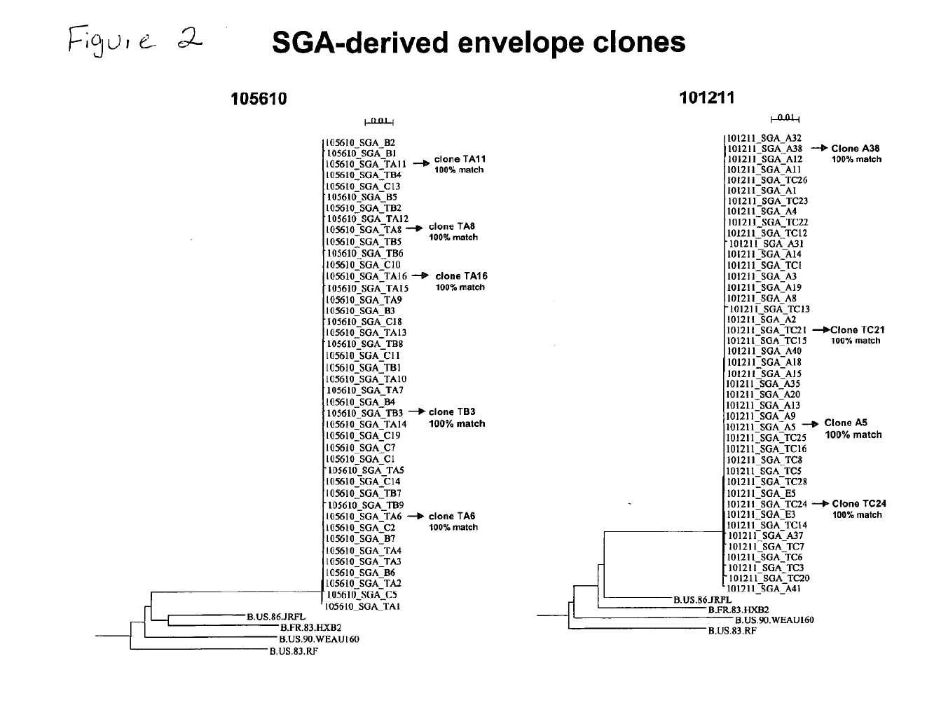

FIG. 2 shows single genome amplification envelop clones derived from 2 AHI patients. Approximately 40 clones were generated per patient and they showed very close homologies with only a few amino acid differences among the clones.

To model viral evolution in early infection, the following assumptions were used for calculating the expected maximum distances for a given number of generations, and for computing simulations of evolution:

At each generation, each cell infects 6 cells

The mutation rate is μ=3.4×10⁻¹

The generation time is 2 days

The Hamming Distance (HD) frequencies follow a Poisson distribution

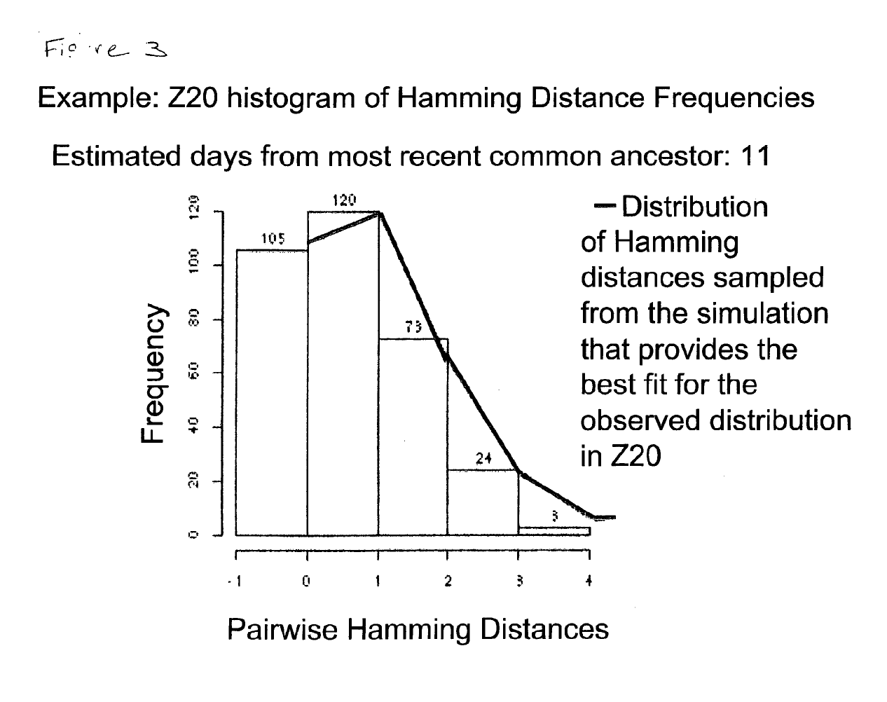

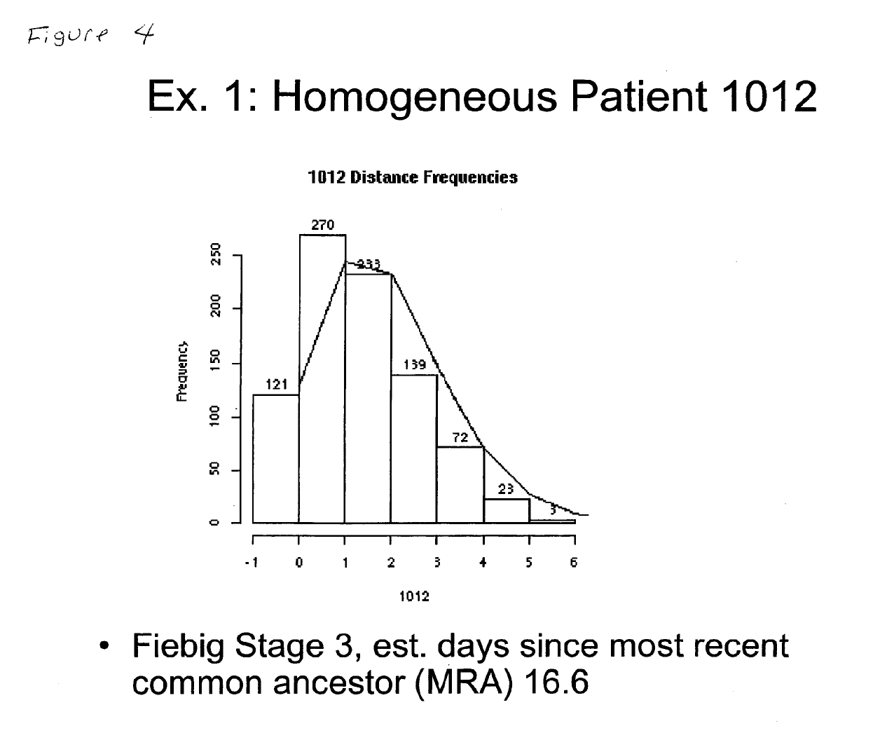

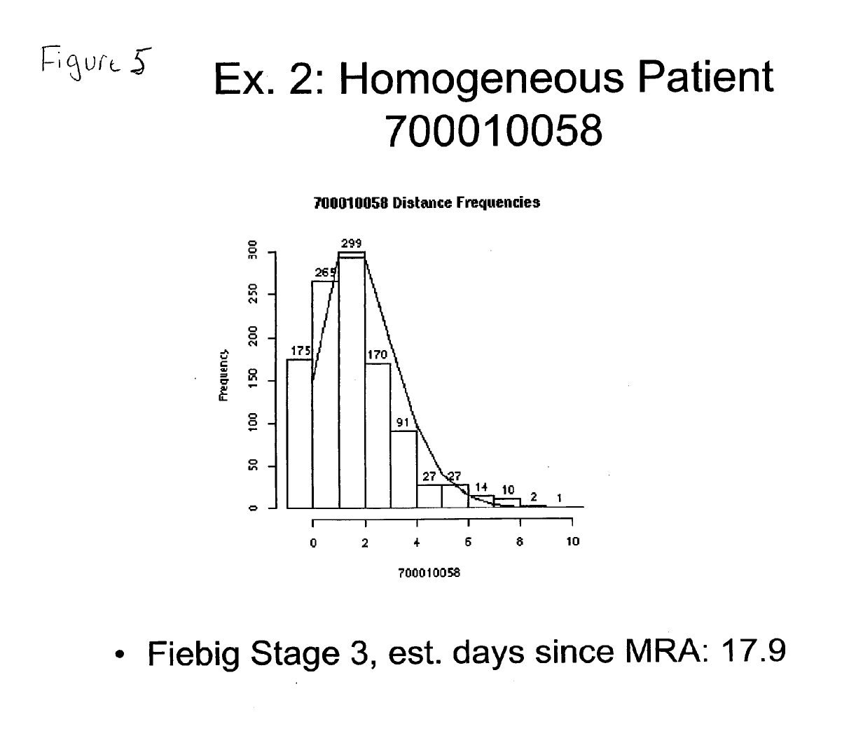

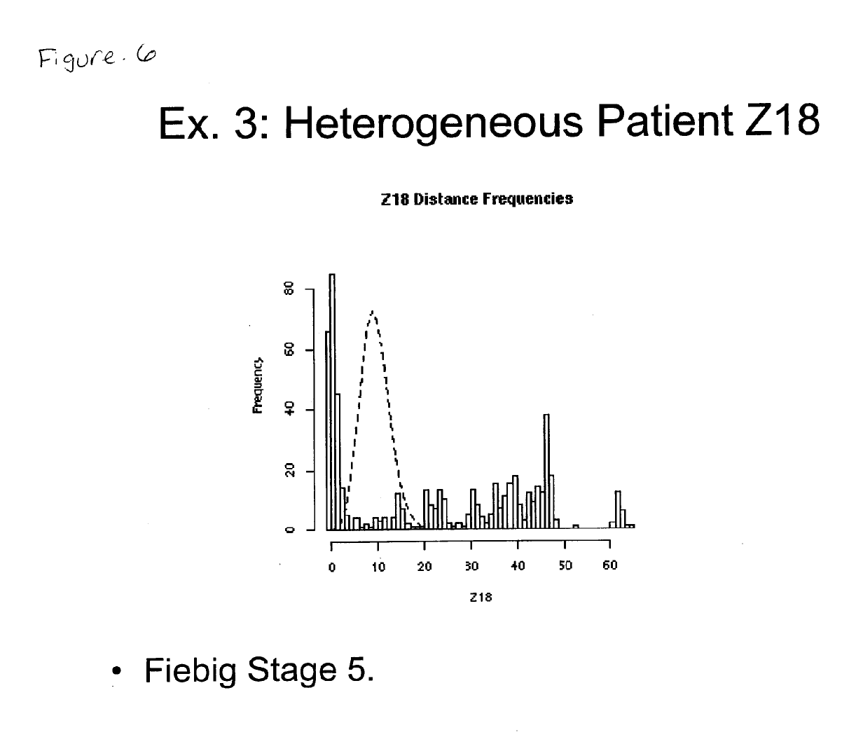

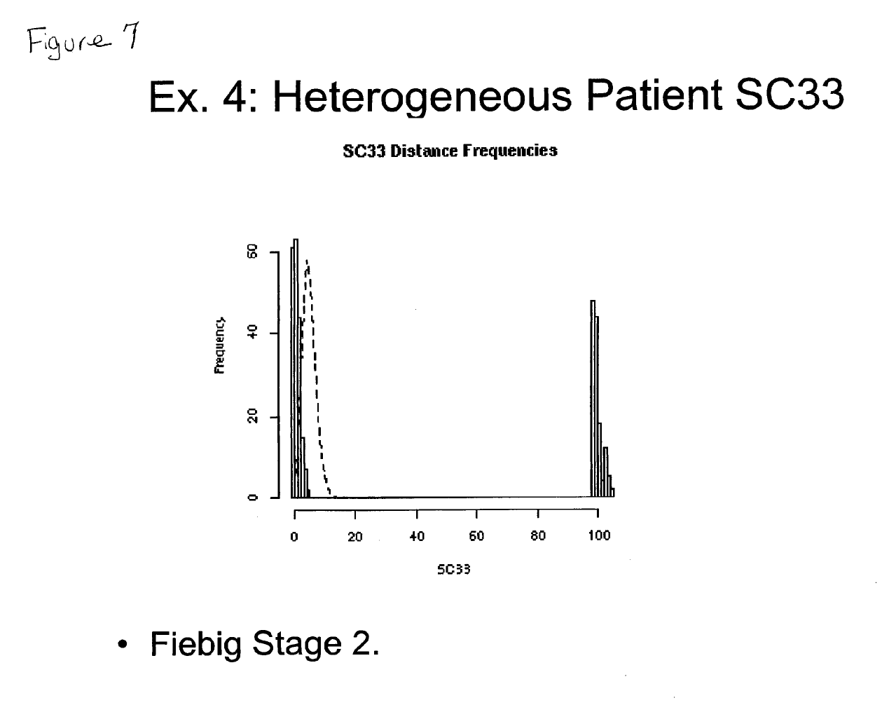

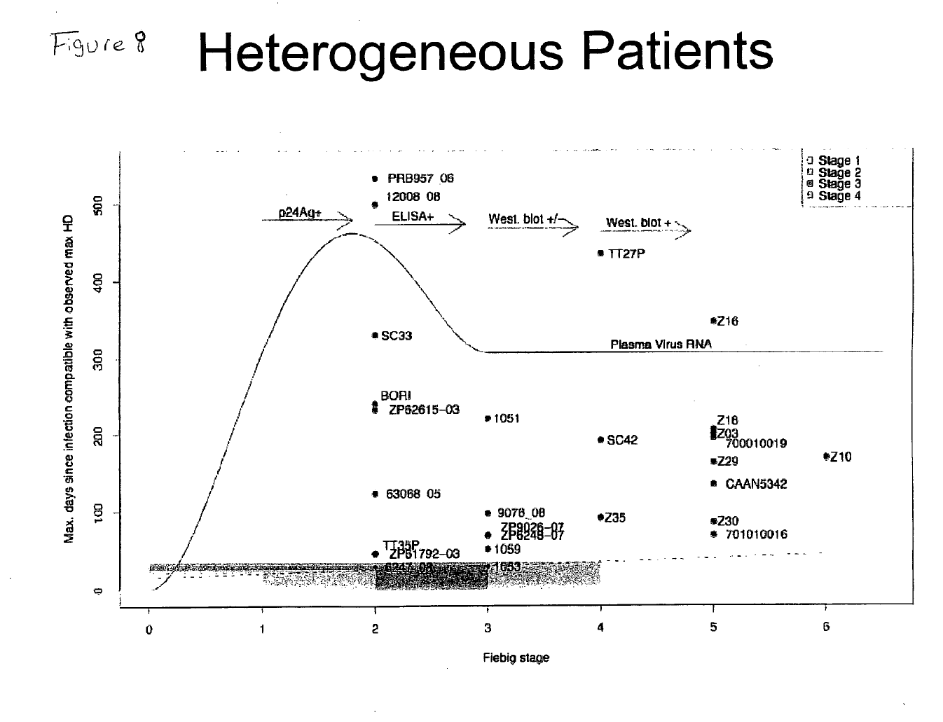

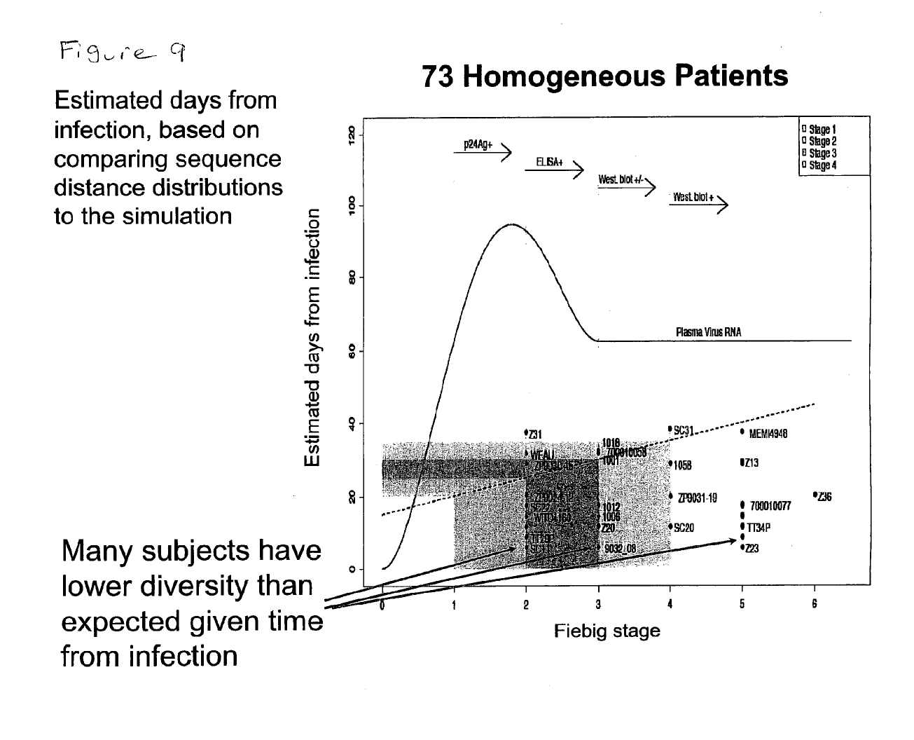

with λ=NBx μ, where NB is the length of the sequence (in bases) FIGS. 3-9 show the results of these analyses.

For the “homogeneous patients” 73/100 samples can be fit well with the model based computer simulation and are consistent with a single virus establishing the infection:
Single peak observed in the Hamming Distance distribution
Relatively homogenous
Estimated days from the MRA within the estimated days from infection based on the Fiebig stage
   However, indications of “selective sweeps” were found in acute infection:
Many samples have an estimated most recent common ancestor (MRA) more recent than than the estimated time from infection
   19/21 stage 1V-VI samples have a most recent common ancestor (MRA)<3 weeks prior
   6/11 stage III samples have an MRA <2 weeks prior
Some samples have a bolus of identical sequences that is unexpected given the rest of the diversity.

A question presented is why might estimated days to the MRAs often be less than the expected days from infection given the Fiebig stage. It is believed that there are two explanations. The model assumptions might give rise to a bias resulting in consistent underestimation of days from the MRA, or, selective sweeps might be real: i.e. serial outgrowth of different lineages may be common during acute infection, resulting from pressures like viral target cell specificity, infiltration of new tissues, or innate immunity prior to HIV specific immune responses.

Given the observed maximum Hamming Distance in a sample, an estimation was made as to how many days it would take to evolve from a shared ancestor to obtain this level of diversity:

Assume 10% extreme selection and 90% neutral drift, per generation step (arbitrary), and

Compute an expected drift per generation for NB that ranges from 2,500 to 3,500.

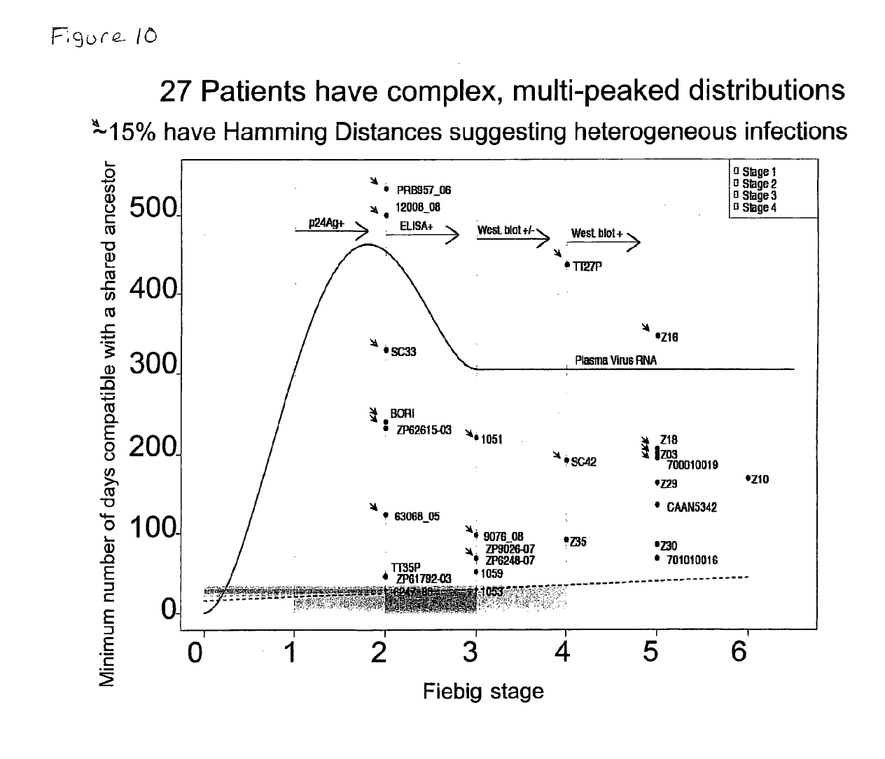

For each patient, an estimate is made of the minimum days it would take to achieve the observed diversity. If this estimate is incompatible with the Fiebig stage, the case is a good candidate for a heterogeneous infection, in which more than one variant was transmitted: ˜15/100 cases. FIG. 10 shows the heterogenous infections using these methods.

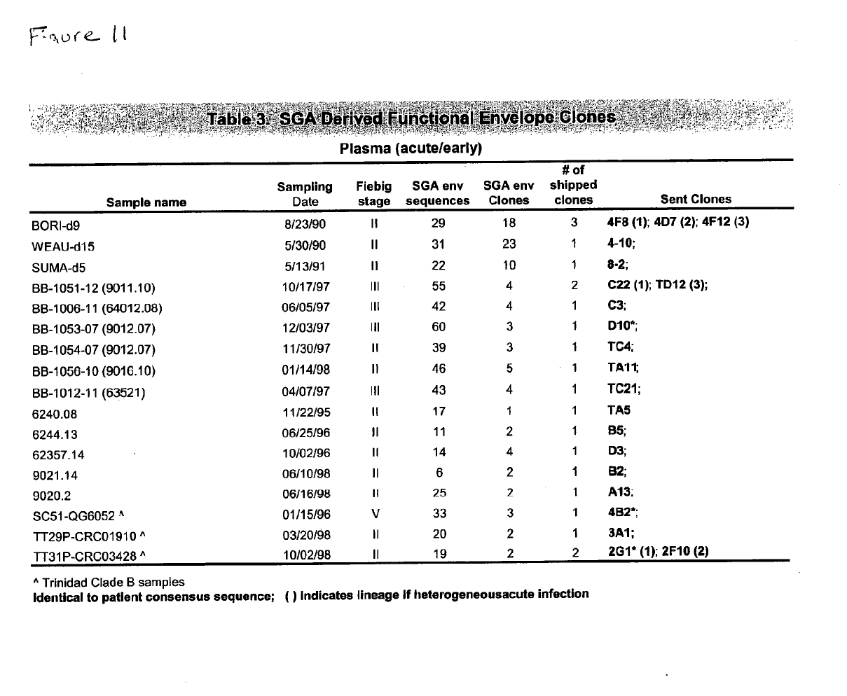

FIG. 11 shows single genome amplification functional envelope clones that have been derived from early acute HIV infection patients that might be used in vaccine development.

Analysis of this Transmitted Virus Dataset for Transmission Virus Signatures

Positive associations require q<0.50 in the test set, and p<0.05 in the validation set. For the initial analyses, two methods of analysis were used:
Mutual information between amino acid positions and acute (or acute+early) sequences and chronic sequence status, and
Patterns of change within the patient consensus tree associated with acute or chronic transmission status.

For mutual information analysis (Korber et al, Proc. Natl. Acad. Sci. USA 90:7176-7180 (1993); Korber et al, AIDS Res. Human Retrovirol. 8:1549-1560 (1992)), a calculation was made of the mutual information between amino acids in a each position and the classification of acute or chronic. The Monte Carlo statistic was used:
Resample each patient with replacement to have equal numbers of sequences per patient before starting,
Shuffle patient classification with 10,000 randomizations, recalculating the mutual information of the randomized data each time, and
Shuffle classifications within clades, to at least partially account for the relatedness (non-independent) samples.

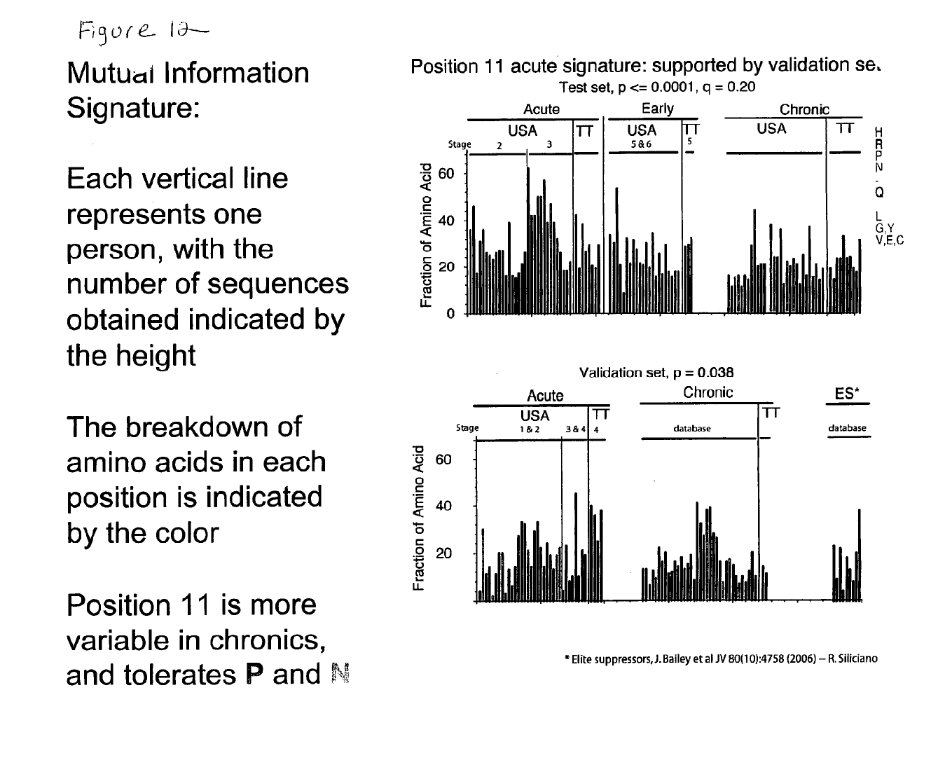

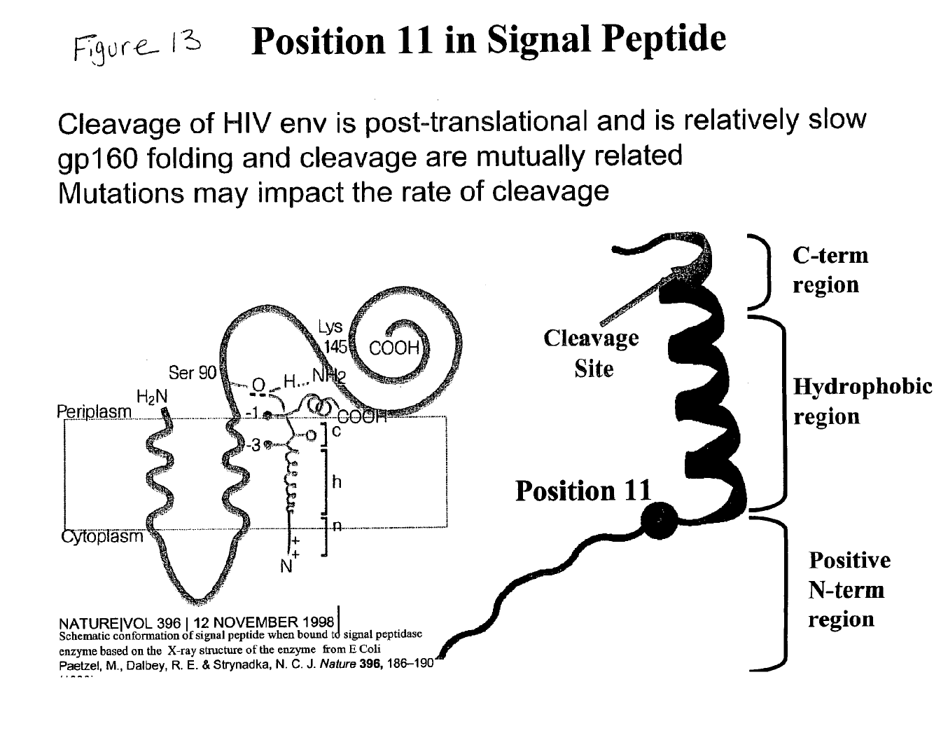

Finally, a determination was made of q-values to contend with multiple tests. FIGS. 12, 13 show a transmitted Env using these methods in the signal sequence of the HIV-1 Env that also overlaps the HIV-1 vpu gene. As shown in FIG. 13, it is hypothesized that this transmitted signature may affect the rate of HIV Env cleavage, and thus provide more Env on the surface of the transmitted virus. Alternatively this mutation may alter the HIV-1 ability to effect Vpu mediated CD4 down modulation (Butticaz et al, J. Virol. 1502-1505 (2007)).

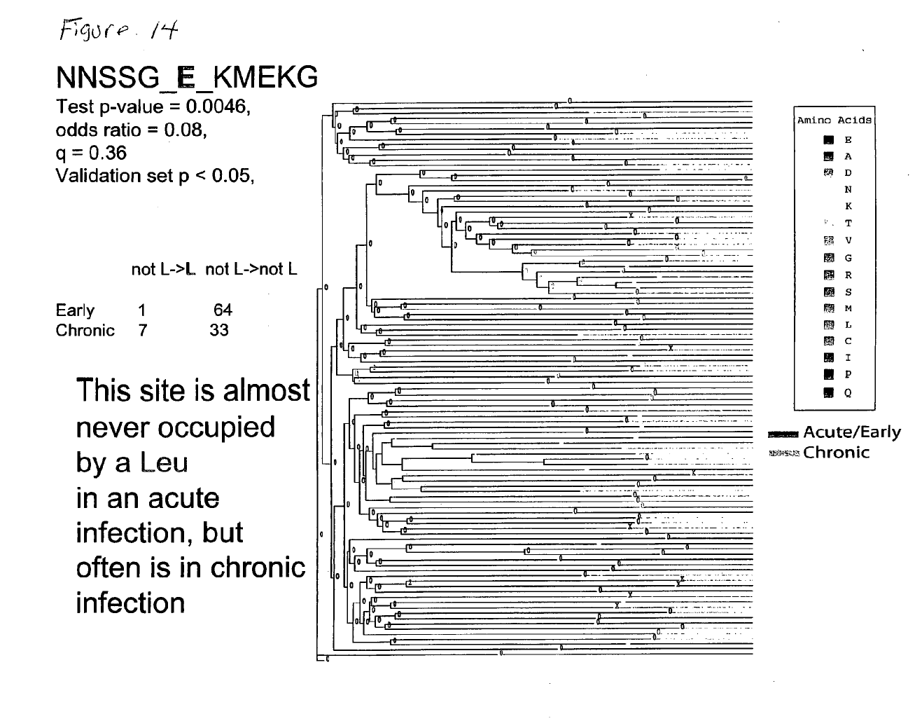

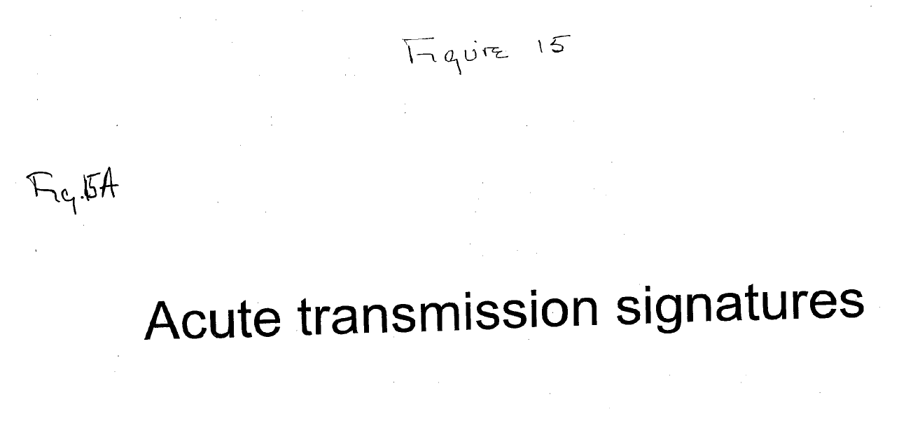

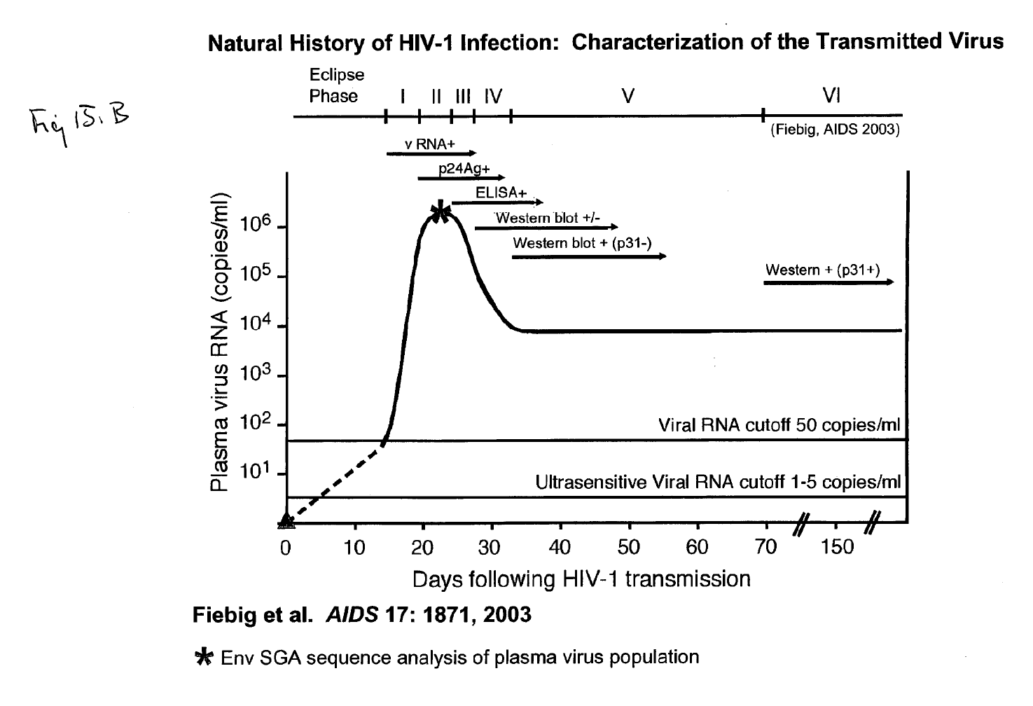

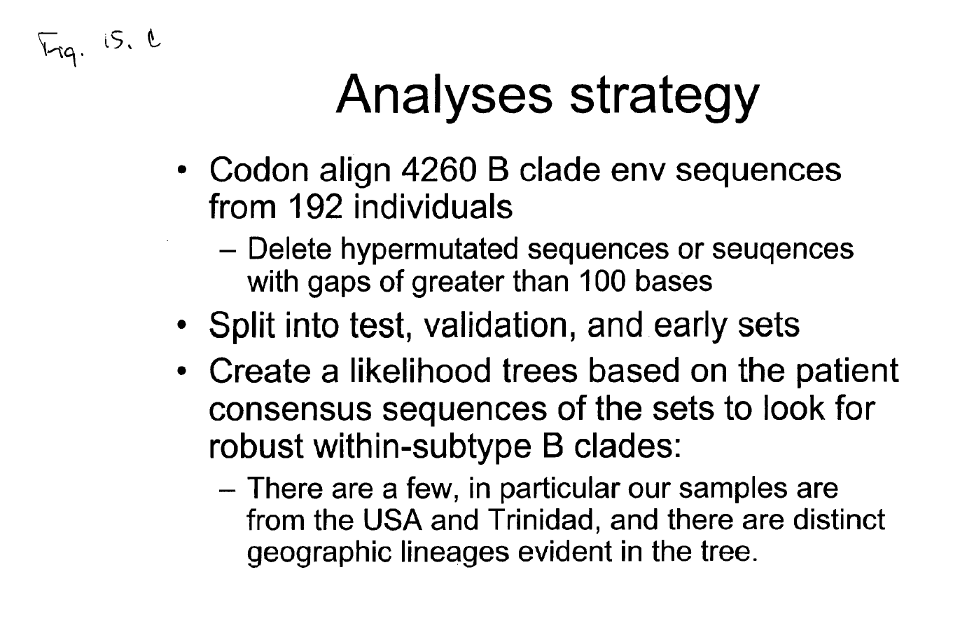

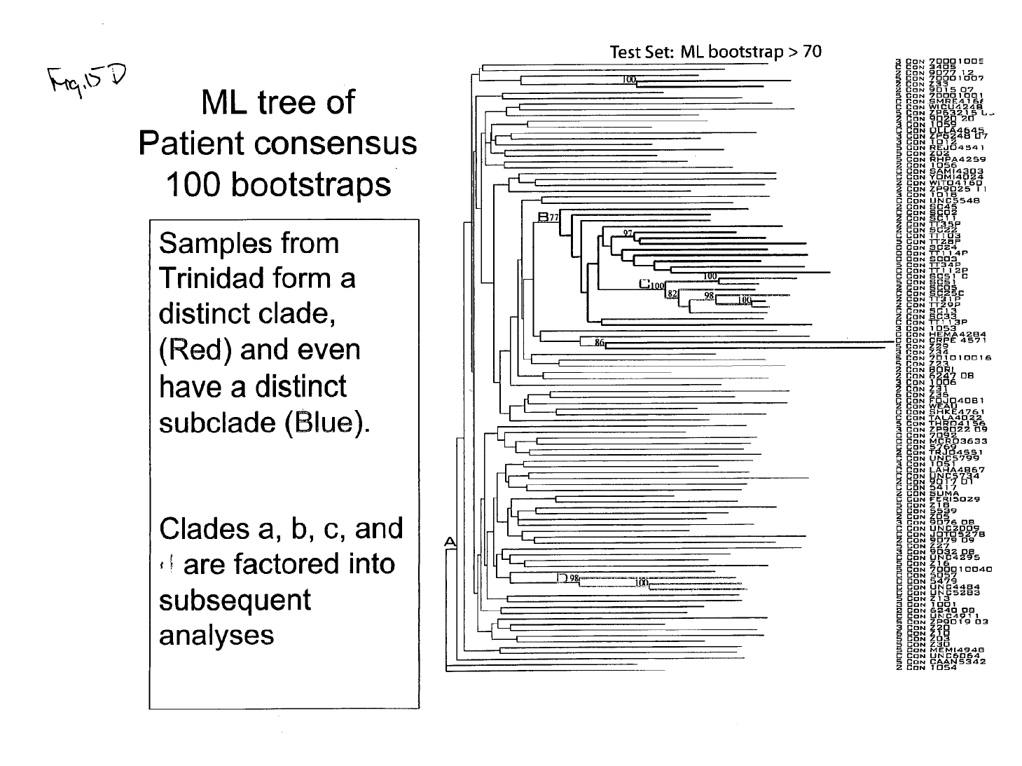

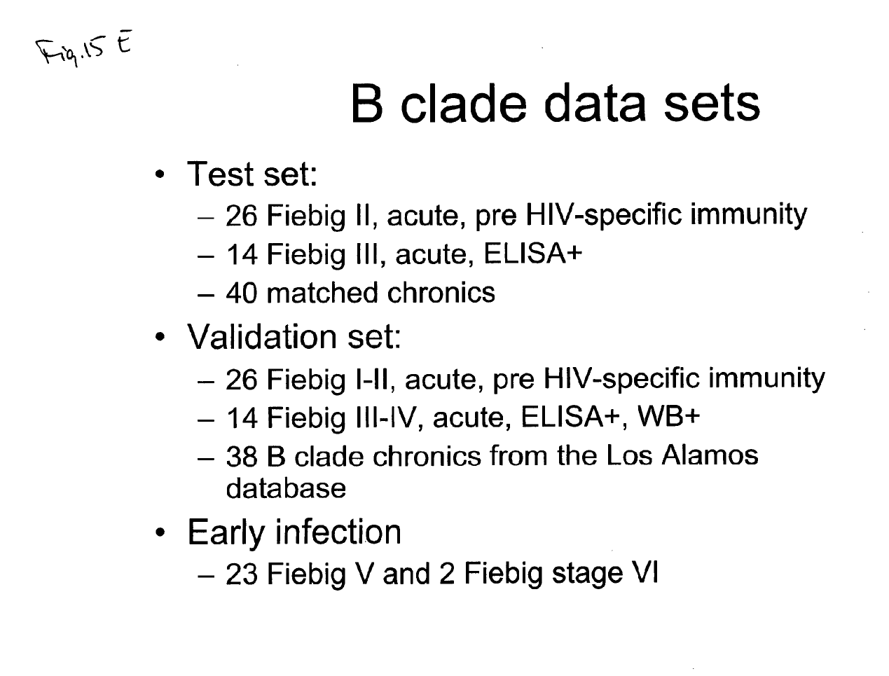

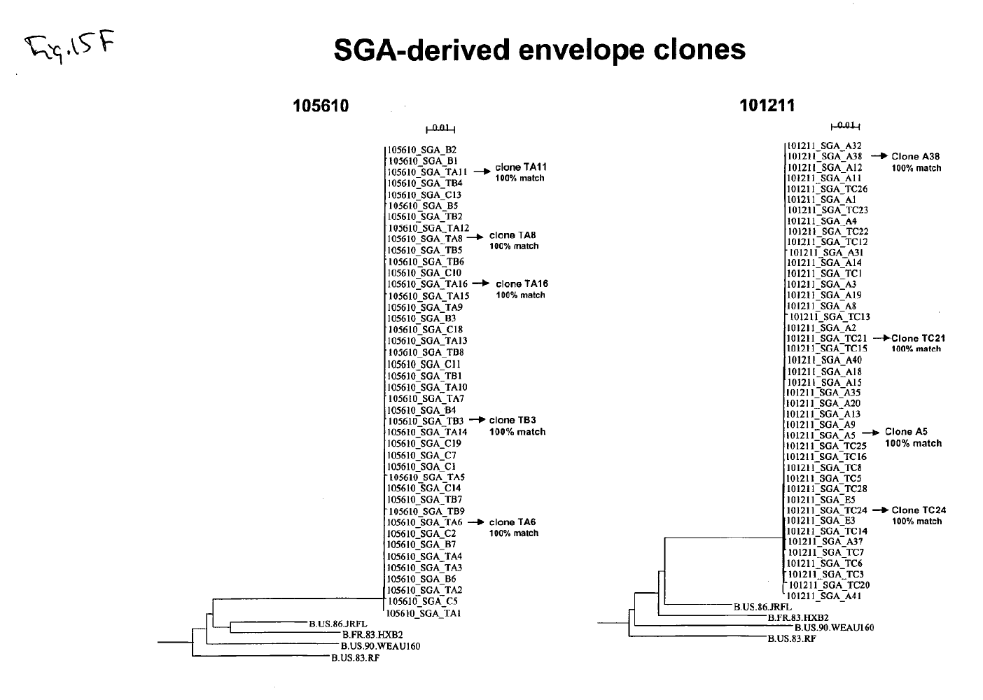

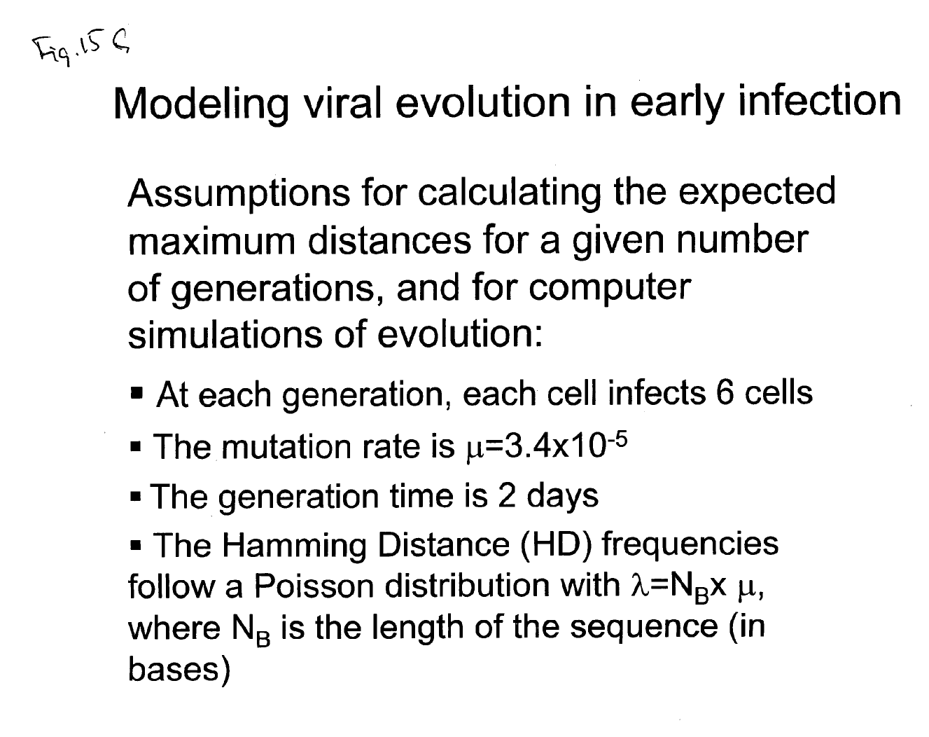

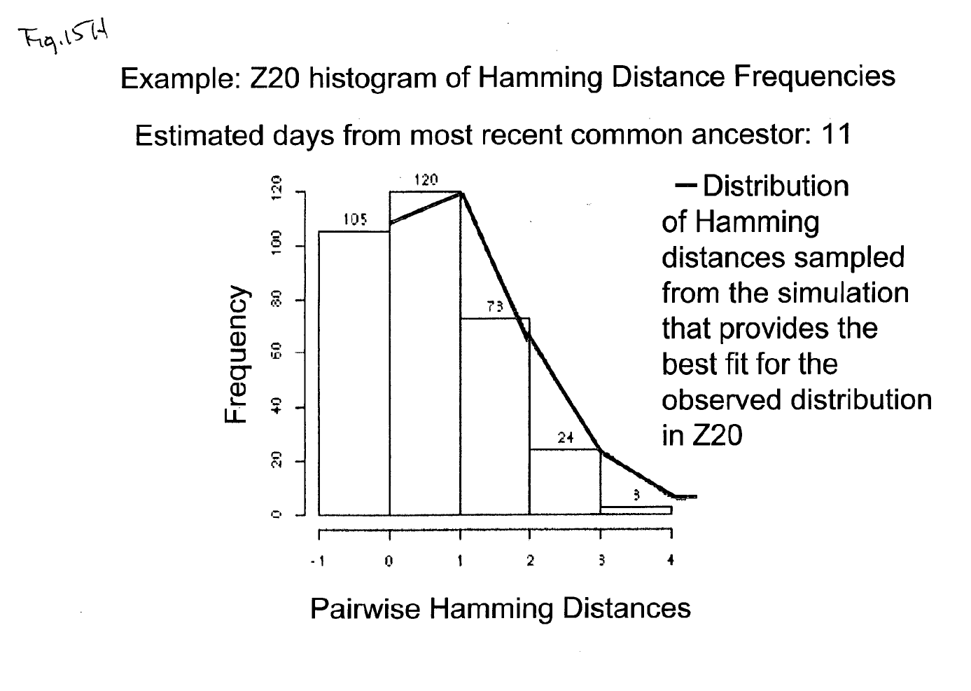

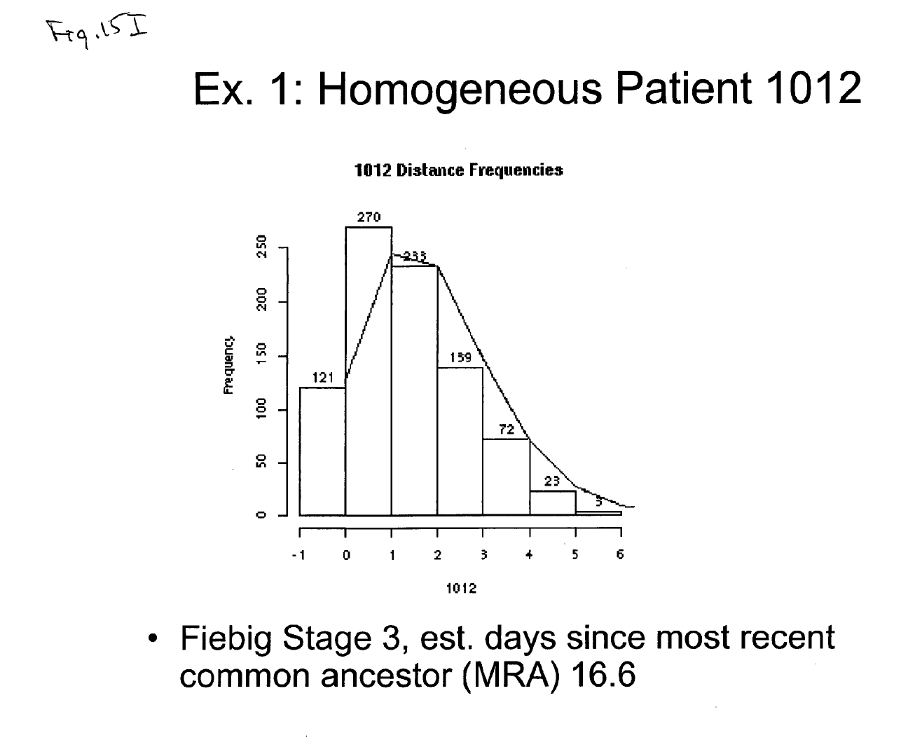

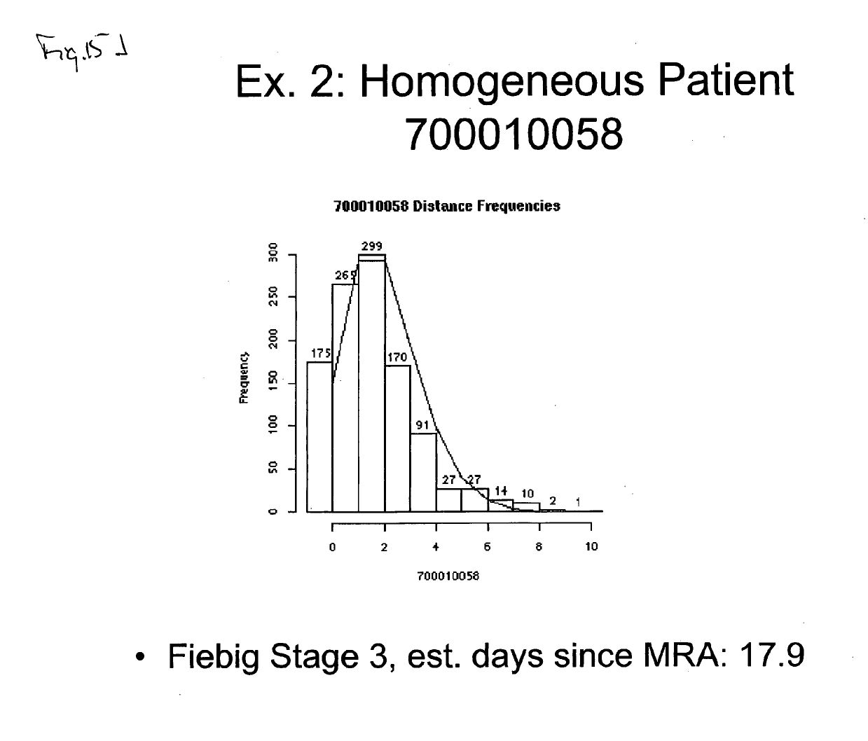

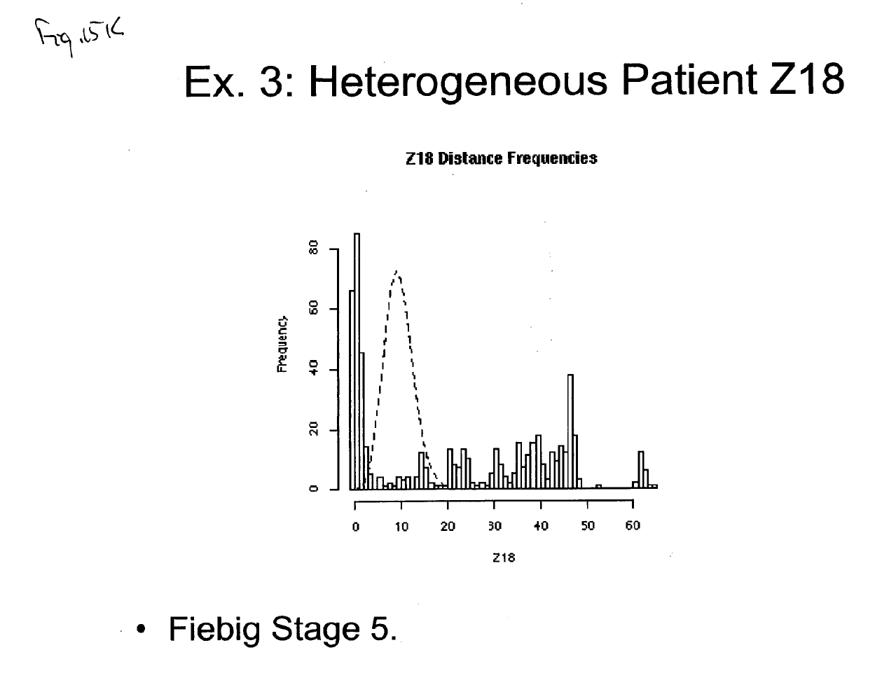

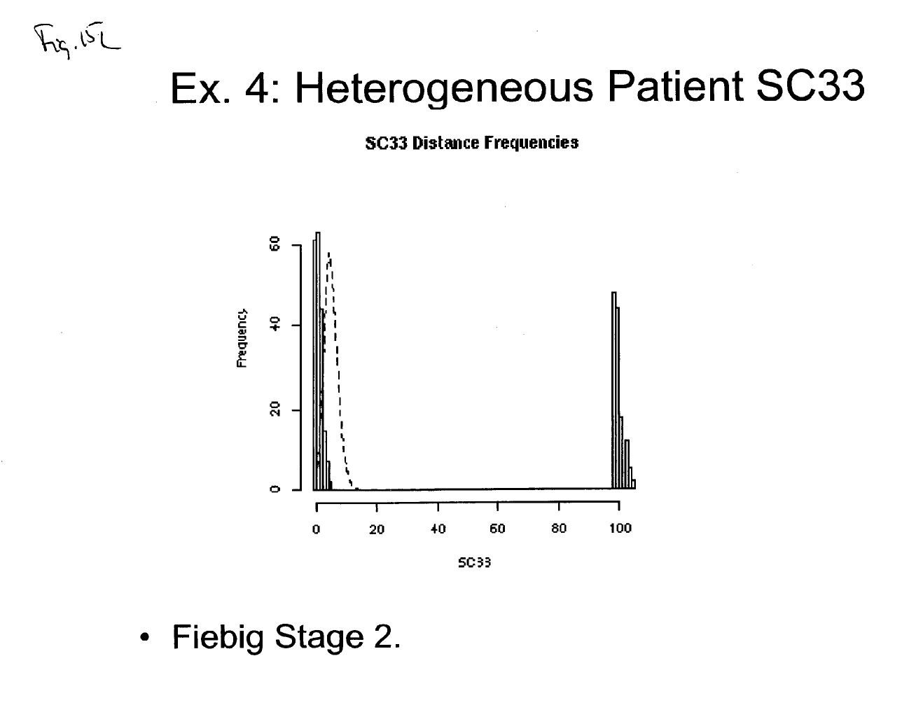

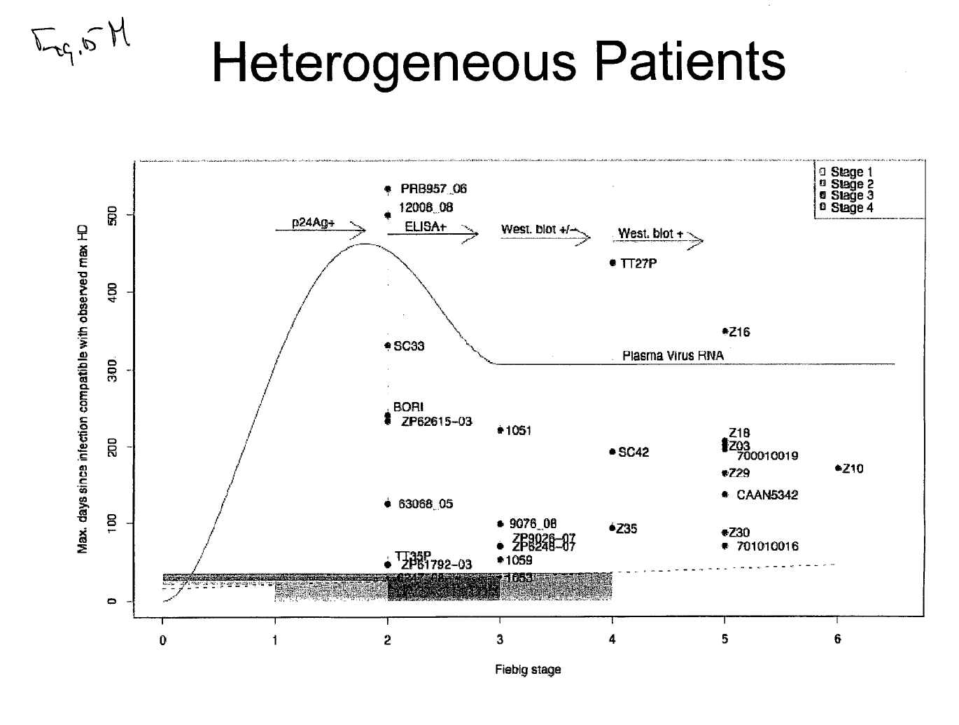

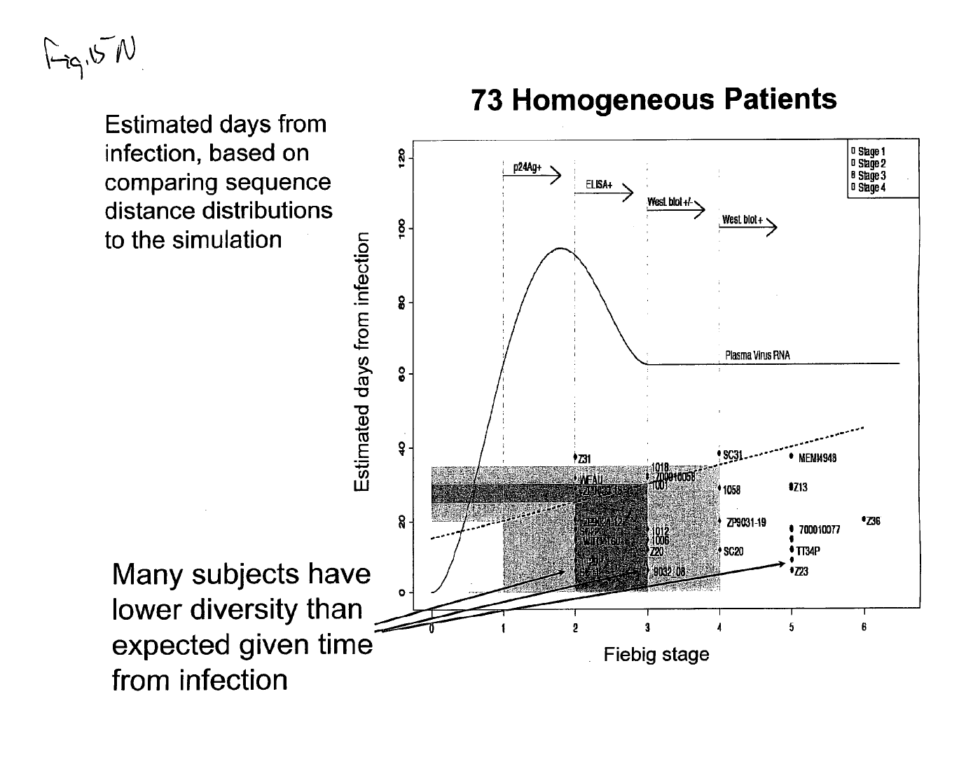

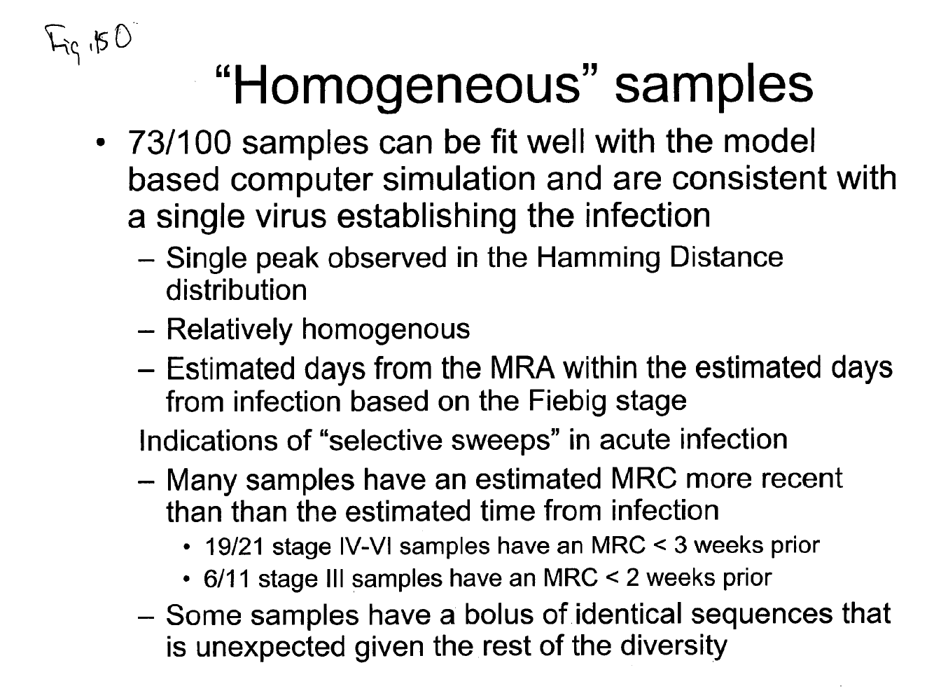

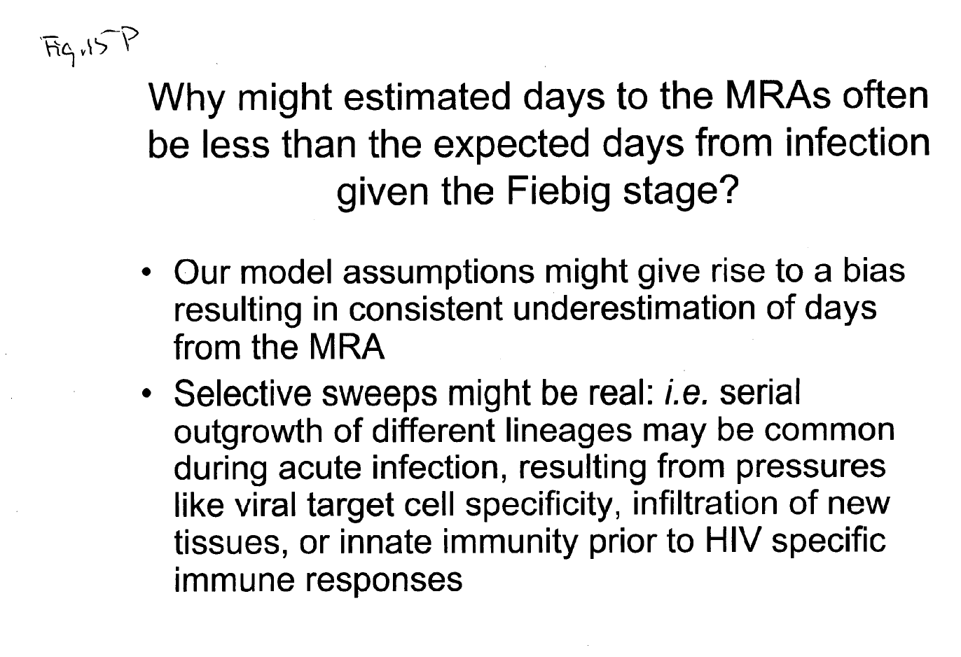

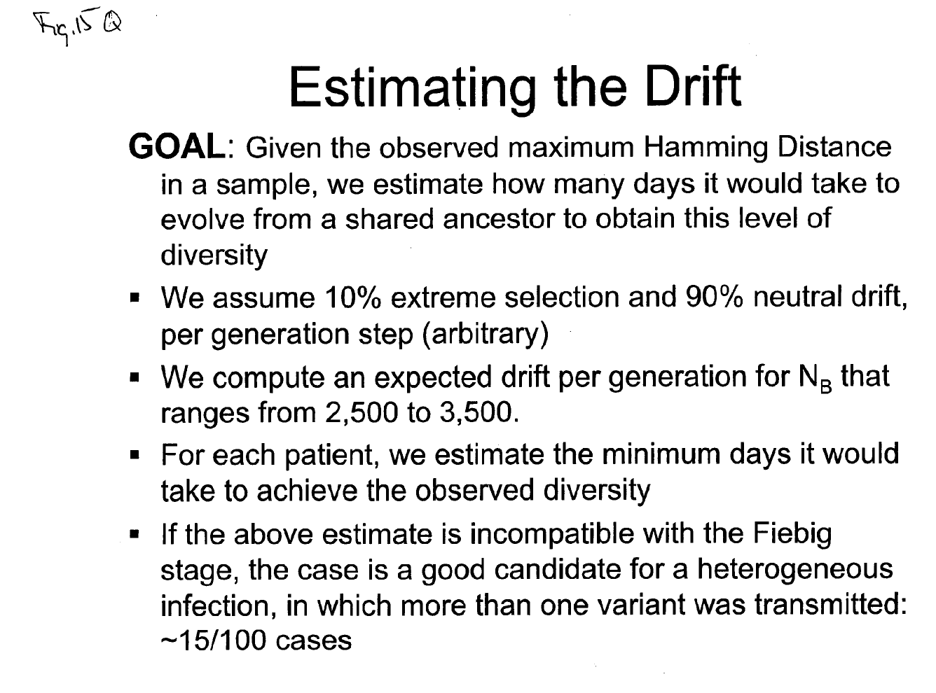

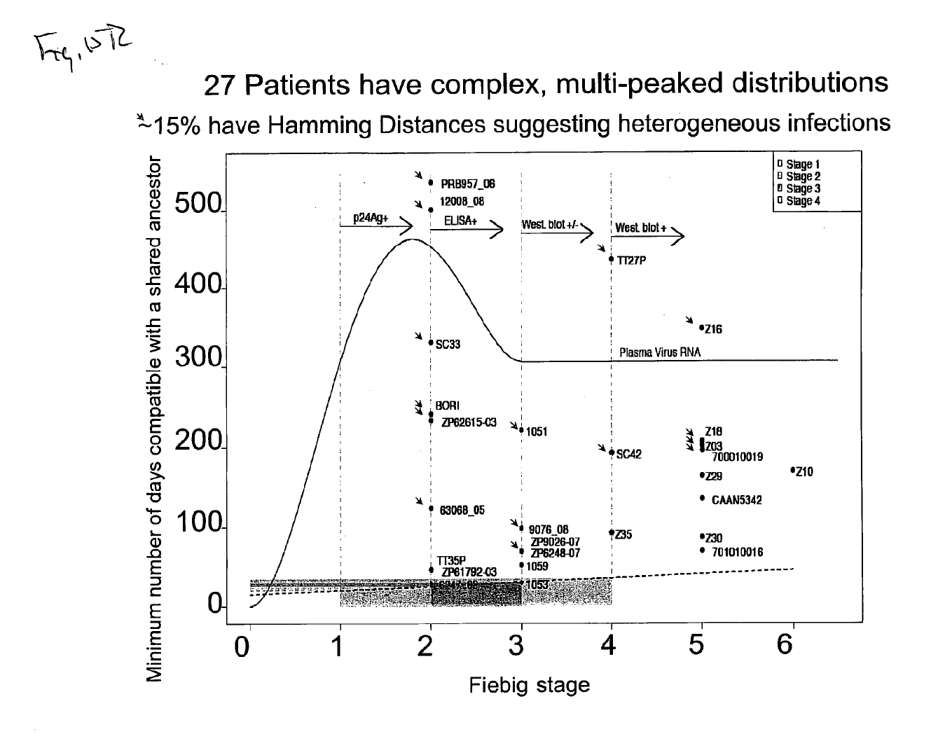

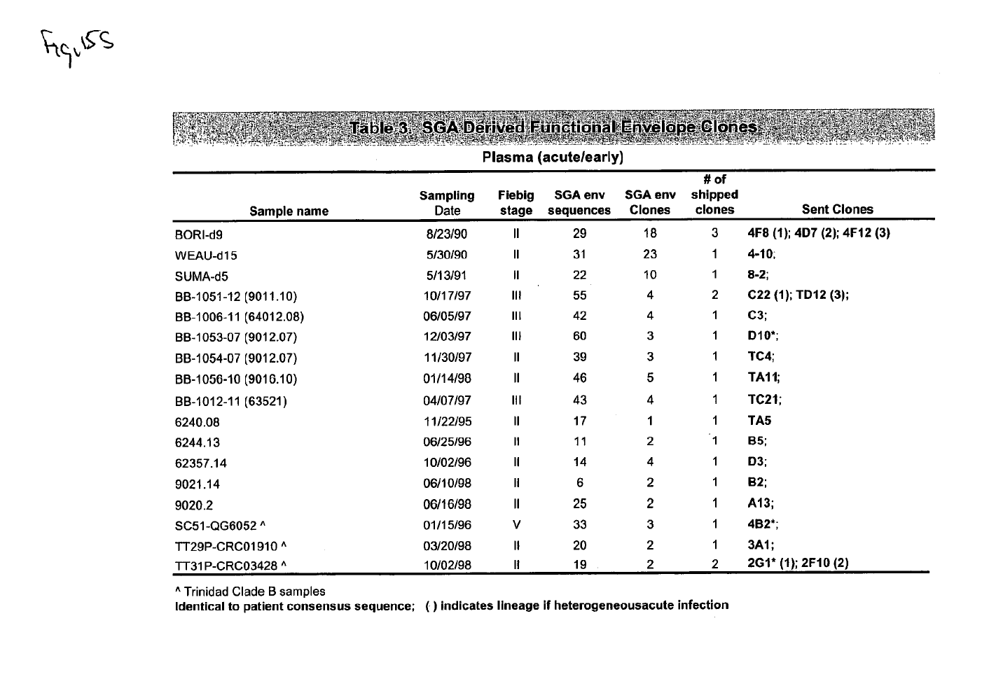

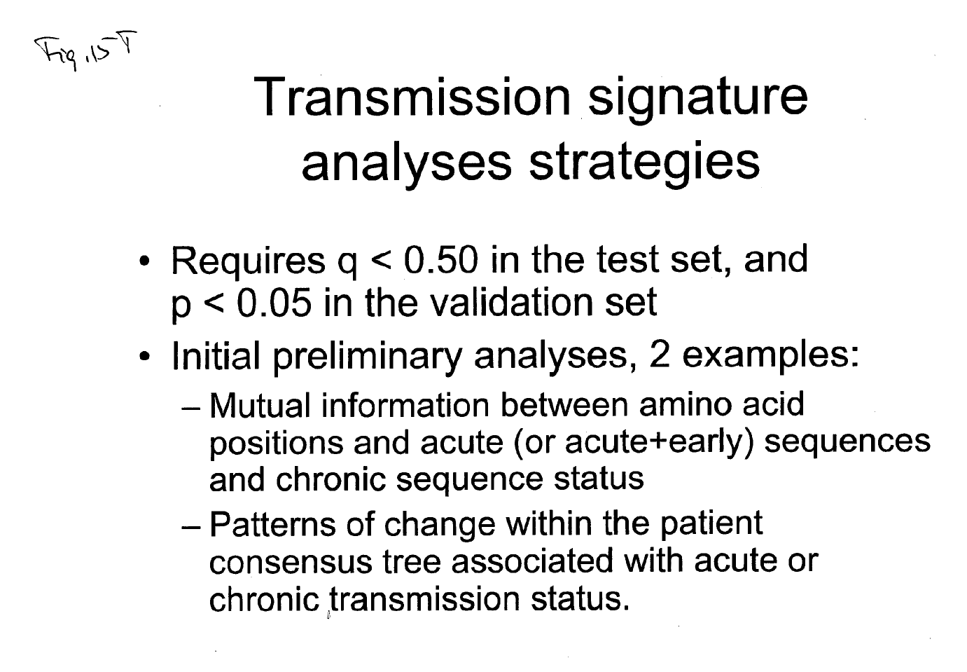

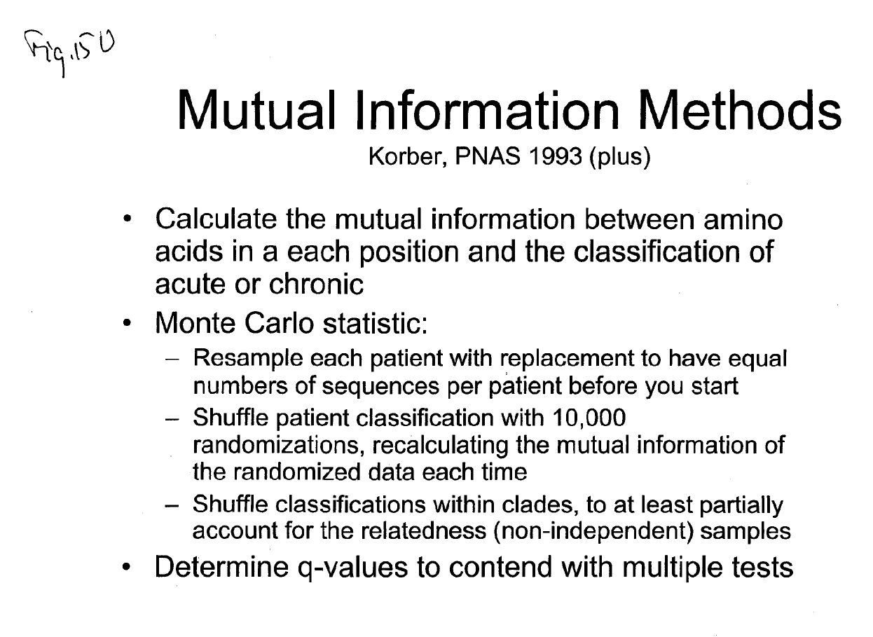

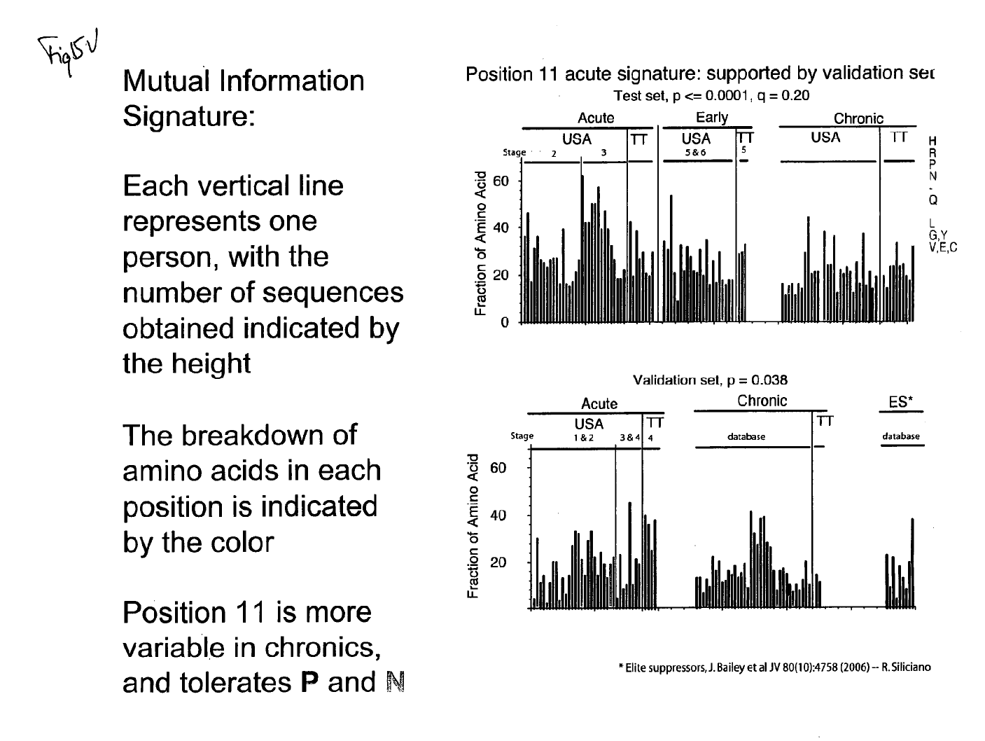

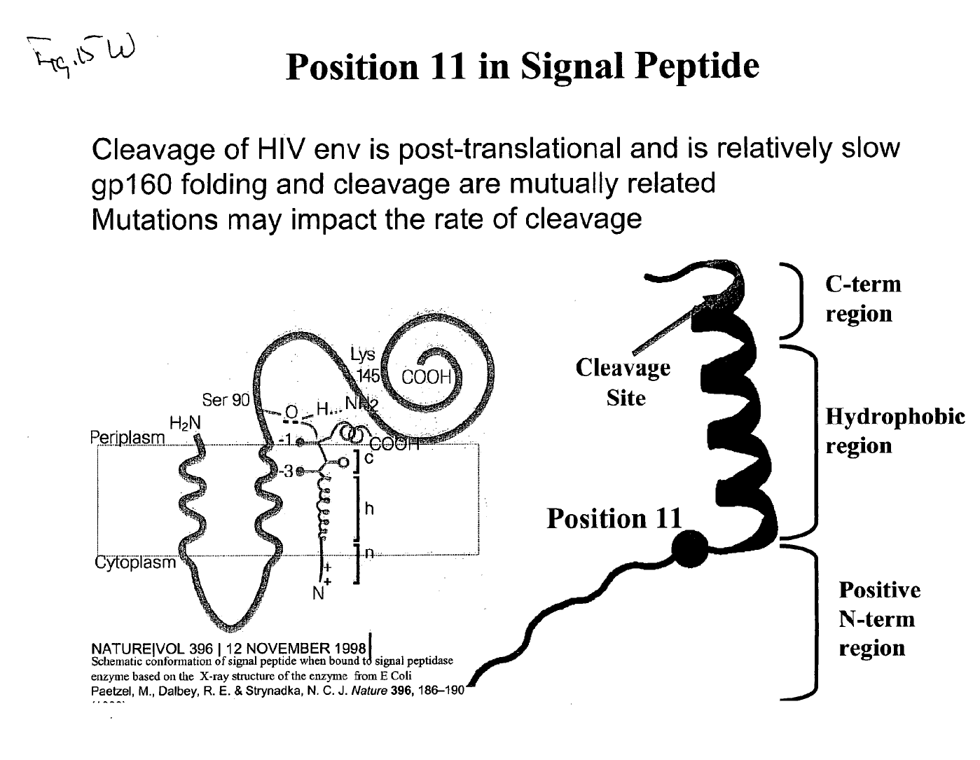

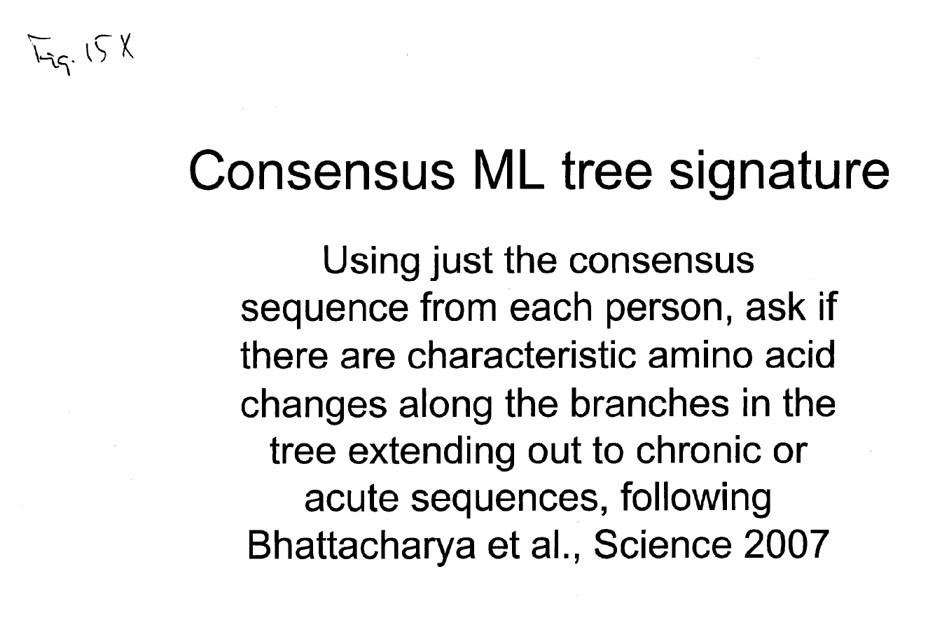

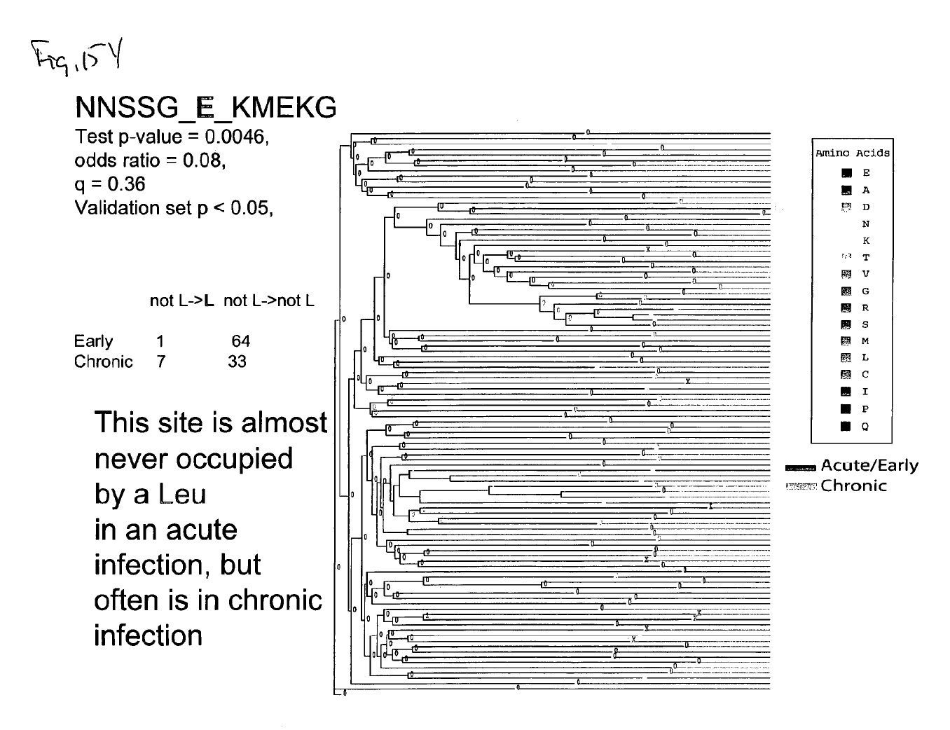

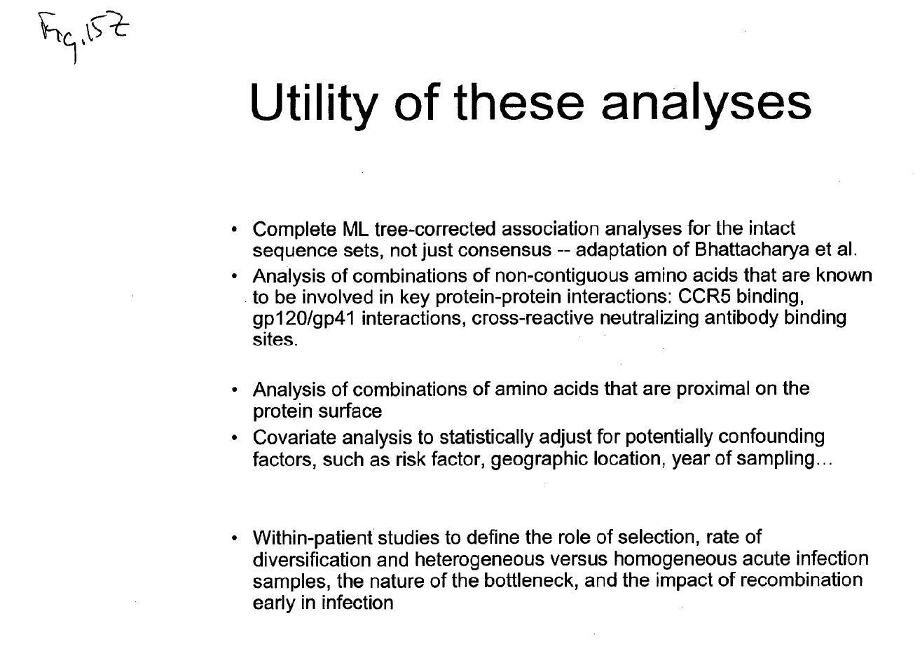

Second, maximum likelihood tree analysis was employed using just the consensus sequence from each person, it was asked whether there are characteristic amino acid changes along the branches in the tree extending out to chronic or acute sequences (see Bhattacharya et al, Science 315:1583-1586 (2007). FIG. 14 shows a transmission signature in the V1 region of HIV-1 Env. It is hypothesized that this signature may affect the neutralization sensitivity of the transmitted HIV virion, and as well may affect exposure of the HIV V3 loop for binding to the CCR5 co-receptor, thus making the transmitted HIV strains more “fit” for transmission.

Another signature was found in the C1 region near to where gp41 is thought to associate with gp120: ENVTE_N_FNMWK amino acid N @ pos 108 in Env gp160. This sequence goes to N in acute transmitted HIV. This mutation may affect stabilization of gp41-gp120 interactions.

Utility of these Analyses

Additional analyses that can be made using the transmitted isolate dataset include:

Complete ML tree-corrected association analyses for the intact sequence sets, not just consensus (adaptation of Bhattacharya et al, Science 315:1583-1586 (2007));

Analysis of combinations of non-contiguous amino acids that are known to be involved in key protein-protein interactions:
CCR5 binding,
gp120/gp41 interactions, and
cross-reactive neutralizing antibody binding sites;

Analysis of combinations of amino acids that are proximal on the protein surface;

Covariate analysis to statistically adjust for potentially confounding factors, such as risk factor, geographic location, year of sampling; and

Within-patient studies to define the role of selection, rate of diversification and heterogeneous versus homogeneous acute infection samples, the nature of the bottleneck, and the impact of recombination early in infection.

All documents and other information sources cited above are hereby incorporated in their entirety by reference.

What is claimed is:

1. A method of inducing an immune response in a mammal comprising administering to said mammal an immunogen comprising a transmitted HIV envelope (Env) sequence signature in an amount sufficient to effect said induction.

2. The method according to claim 1 wherein said transmitted HIV Env sequence signature is present in a consensus Env.

3. The method according to claim 2 wherein said consensus Env is a group M consensus Env.

4. The method according to claim 1 wherein said transmitted HIV Env sequence signature affects the rate of HIV Env cleavage or alters the HIV ability to effect Vpu-mediated CD4 down modulation.

5. The method according to claim 1 wherein said transmitted HIV Env sequence signature is in the signal sequence of HIV Env.

6. The method according to claim 1 wherein said transmitted HIV Env sequence signature is in the VI region of HIV-Env.

7. The method according to claim 6 wherein said transmitted HIV Env sequence signature affects neutralization sensitivity of a transmitted HIV virion or exposure of the HIV V3 loop for binding to the CCRS co-receptor.

8. The method according to claim 1 wherein said transmitted HIV Env sequence signature is in the C1 region of HIV ENV.

9. The method according to claim 8 wherein said transmitted HIV Env sequence signature affects stabilization of gp41-gp120 interactions.

10. The method according to claim 1 wherein said mammal is a human.

11. A composition comprising a mixture of transmitted HIV Env sequence signatures and a carrier.
